(19) United States
(12) Patent Application Publication
(10) Pub. No.: US 2021/0287767 A1
(43) Pub. Date: Sep. 16, 2021

(54) MOBILE TERMINAL, COMPUTER READABLE RECORDING MEDIUM AND WALLET SYSTEM

(71) Applicant: TOYOTA JIDOSHA KABUSHIKI KAISHA, Toyota-shi (JP)

(72) Inventors: Hiroto TOMIDA, Tokyo (JP); Tae SUGIMURA, Miyoshi-shi (JP); Ryuta YANAGISAWA, Ichikawa-shi (JP); Takuya TORIU, Tokyo (JP); Daisuke KOMIYAMA, Tokyo (JP); Zejun MEI, Fujimi-shi (JP); Kazuhiko YAMAGUCHI, Nagoya-shi (JP); Honami MORI, Nagoya-shi (JP)

(73) Assignee: TOYOTA JIDOSHA KABUSHIKI KAISHA, Toyota-shi (JP)

(21) Appl. No.: 17/164,008

(22) Filed: Feb. 1, 2021

(30) Foreign Application Priority Data
Mar. 12, 2020 (JP) 2020-043491

(51) Int. Cl. G16H 10/60; G06F 3/0481; G16H 20/10; G06Q 20/14; G06Q 20/36

(52) U.S. Cl. CPC G16H 10/60; G06F 3/0481; H04W 88/02; G06Q 20/14; G06Q 20/367; G16H 20/10

(57) ABSTRACT

A mobile terminal includes: a display; and a processor including hardware, the processor being configured to acquire progress information indicating progress of when a user visits a medical institution from a first server provided at the medical institution, and control the display to display the progress information.

The present application claims priority to and incorporates by reference the entire contents of Japanese Patent Application No. 2020-043491 filed in Japan on Mar. 12, 2020.

### BACKGROUND

The present disclosure relates to a mobile terminal, a computer readable recording medium and a wallet system.

In JP 2017-151732 A, a system that centrally manages user information by managing information of a user (patient), information for a medical institution to identify the user, and a social security and tax number of the user in association with each other is disclosed.

### SUMMARY

There is a need for a mobile terminal, a computer readable recording medium and a wallet system that improves convenience for a user who uses a medical institution.

According to one aspect of the present disclosure, there is provided a mobile terminal including: a display; and a processor including hardware, the processor being configured to acquire progress information indicating progress of when a user visits a medical institution from a first server provided at the medical institution, and control the display to display the progress information.

### BRIEF DESCRIPTION OF THE DRAWINGS

FIG. 1 is a view schematically illustrating a configuration of a wallet system according to an embodiment;

FIG. 2 is a block diagram illustrating details of each component of the wallet system;

FIG. 3 is a view illustrating a first example in which progress information is displayed on a user terminal in the wallet system;

FIG. 4 is a view illustrating a second example in which progress information is displayed on the user terminal in the wallet system;

FIG. 5 is a view illustrating a third example in which progress information is displayed on the user terminal in the wallet system;

FIG. 6 is a view illustrating an example in which a prescription is displayed on the user terminal in the wallet system;

FIG. 7 is a view illustrating a fourth example in which progress information is displayed on the user terminal in the wallet system;

FIG. 8 is a view illustrating a fifth example in which progress information is displayed on the user terminal in the wallet system; and

FIG. 9 is a flowchart illustrating an example of a progress information displaying method of a medical institution which method is executed by the wallet system.

### DETAILED DESCRIPTION

A mobile terminal, a computer readable recording medium storing a wallet program, and a wallet system according to an embodiment of the present disclosure will be described with reference to the drawings. Note that components in the following embodiment include what may be easily replaced by those skilled in the art or what is substantially the same.

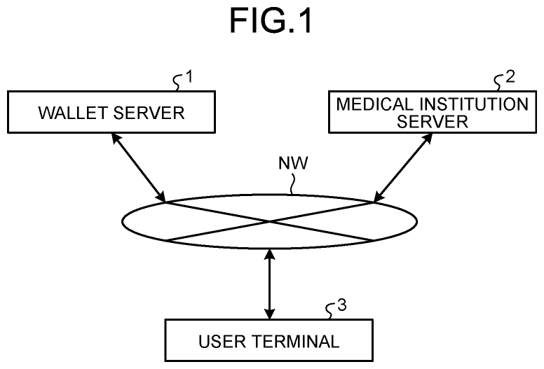

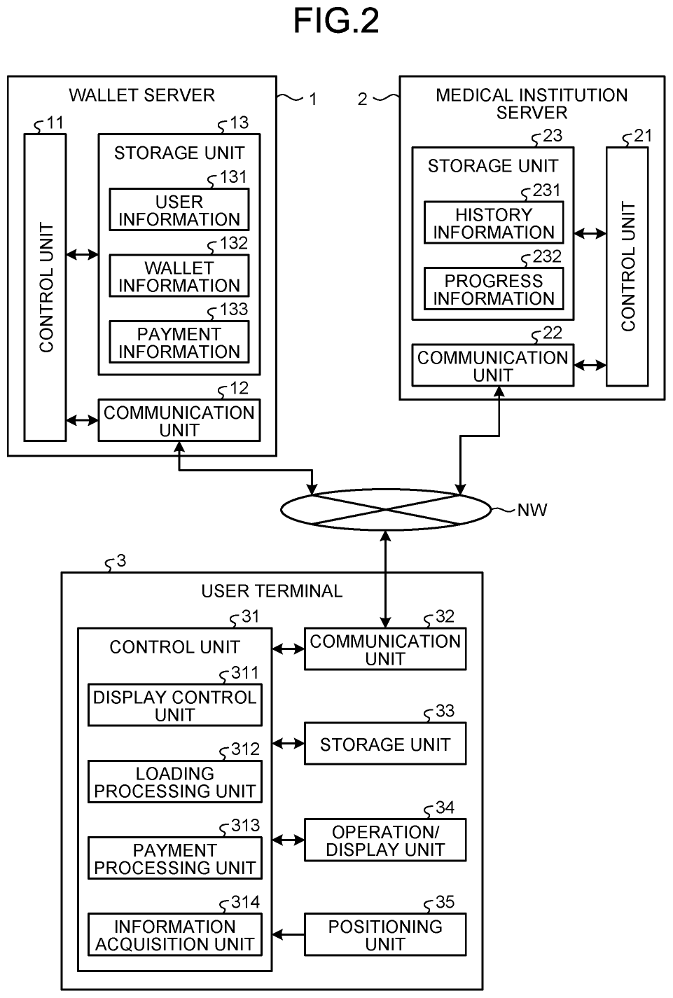

A configuration of a wallet system will be described with reference to FIG. 1 and FIG. 2. As illustrated in FIG. 1, the wallet system includes a wallet server 1, a medical institution server 2, and a user terminal 3. In the wallet system, the wallet server 1, the medical institution server 2, and the user terminal 3 are configured in a manner of being able to communicate with each other through a network NW. This network NW includes, for example, an Internet network, a mobile phone network, and the like. Note that in the drawing, only one medical institution server 2 and one user terminal 3 are illustrated. However, there may be a plurality of these.

The wallet server (second server) 1 is a server to manage the wallet system. This wallet server 1 is realized by a general-purpose computer such as a workstation or a personal computer. Moreover, the wallet server 1 is managed by an administrator (provider) of the wallet system. As illustrated in FIG. 2, the wallet server 1 includes a control unit (second processor) 11, a communication unit 12, and a storage unit 13.

More specifically, the control unit 11 includes a processor including a central processing unit (CPU), a digital signal processor (DSP), a field-programmable gate array (FPGA), and the like, and a memory (main storage unit) including a random access memory (RAM), a read only memory (ROM), and the like. The control unit 11 realizes a function that meets a predetermined purpose by loading and executing a program, which is stored in the storage unit 13, in a work area of the main storage unit and controlling each configuration unit and the like through execution of the program.

The communication unit 12 includes, for example, a local area network (LAN) interface board, a wireless communication circuit for wireless communication, and the like. The communication unit 12 is connected to the network NW that is a public communication network, such as the Internet. Then, the communication unit 12 communicates with the medical institution server 2 and the user terminal 3 by being connected to the network NW.

The storage unit 13 includes recording media such as an erasable programmable ROM (EPROM), a hard disk drive (HDD), and a removable medium. Examples of the removable media include a universal serial bus (USB) memory, and disc recording media such as a compact disc (CD), a digital versatile disc (DVD), and a Blu-ray (registered trademark) disc (BD). The storage unit 13 may store an operating system (OS), various programs, various tables, various databases, and the like. Moreover, the storage unit 13 stores user information 131, wallet information 132, and payment information 133 of each user of the wallet system.

The user information 131 is information related to a user who uses the wallet system. Examples of the user information 131 include a user ID, password, user name, user contact information (such as address, telephone number, and email address), and the like of a user who has registered in advance to use the wallet system. Note that the user ID and password in the user information 131 are used for authentication processing such as a login to the wallet server 1.

The wallet information 132 is information related to a wallet (electronic wallet) of the wallet system. The wallet indicates a virtual deposit/withdrawal account of electronic money. The wallet information 132 includes, for example, a user ID, a balance of electronic money, a loading history of the electronic money, a usage history of the electronic money, and the like.

The payment information 133 is information related to payment in the wallet system. The payment information 133 includes a user ID, a payment means used for payment (such as electronic money payment (contactless payment), scan payment, or code payment), a payment history, and the like.

The medical institution server 2 is a server provided in a medical institution. This medical institution server 2 is realized by a general-purpose computer such as a workstation or a personal computer.

As illustrated in FIG. 2, the medical institution server (first server) 2 includes a control unit (first processor) 21, a communication unit 22, and a storage unit 23. The control unit 21, the communication unit 22, and the storage unit 23 are physically similar to the control unit 11, the communication unit 12, and the storage unit 13. History information 231 and progress information 232 are stored in the storage unit 23.

The history information 231 is information related to a medical institution consultation history of each user. The history information 231 includes, for example, a medical treatment history of a user who visits a medical institution, a prescription history of a medicine for the user, a medical fee payment (payment) history of the user, and the like.

The progress information 232 is information related to progress of when a user visits a medical institution. The progress information 232 includes, for example, information indicating whether the user is undergoing a medical examination, status information indicating whether preparation of a prescription is completed, status information indicating whether calculation of a medical fee is completed, status information indicating whether a payment of the medical fee is completed, prescription data, medical fee data, and the like. For example, when the user visits the medical institution, the control unit 21 of the medical institution server 2 outputs the latest progress information 232 to the user terminal 3 every time a status is changed.

The user terminal 3 is a mobile terminal used by a user. The “user” in the present disclosure refers to a patient who uses the wallet system and visits a medical institution. Through the user terminal 3, the user may make an appointment at a medical institution, confirm progress of a medical treatment and various procedures at the medical institution, confirm and change a prescription, pay a medical fee, and confirm a medical statement and receipt. Examples of the user terminal 3 include a smartphone, a mobile phone, a tablet terminal, a wearable computer, and the like owned by the user.

As illustrated in FIG. 2, the user terminal 3 includes a control unit (third processor) 31, a communication unit 32, a storage unit 33, an operation/display unit (display) 34, and a positioning unit 35. The control unit 31, the communication unit 32, and the storage unit 33 are physically similar to the control unit 11, the communication unit 12, and the storage unit 13. The control unit 31 functions as a display control unit 311, a loading processing unit 312, a payment processing unit 313, and an information acquisition unit 314 through execution of a program.

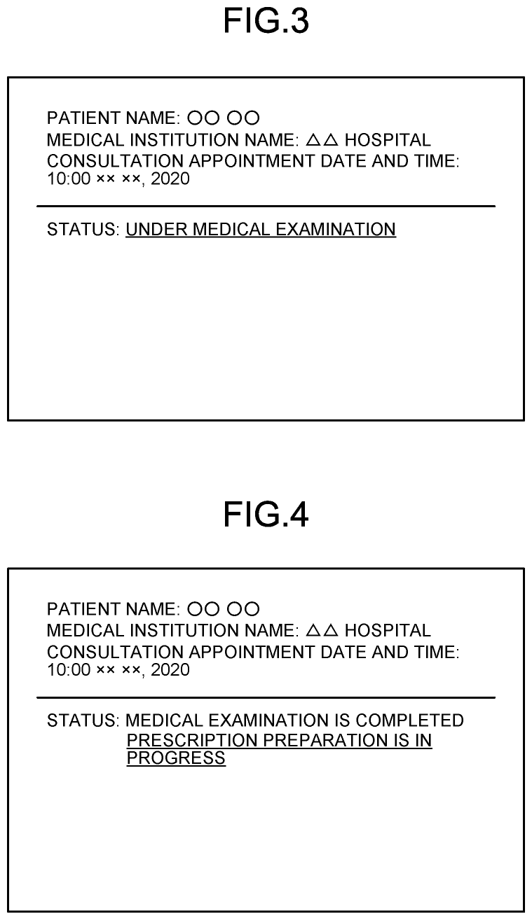

The display control unit 311 controls display contents on the operation/display unit 34. The display control unit 311 causes the operation/display unit 34 to display predetermined information based on an operation (such as touch operation or flick operation) by the user. Examples of the information displayed on the operation/display unit 34 by the display control unit 311 include a member registration screen of when the user registers as a member of the wallet system, a login screen of when a login to the wallet system is performed, a loading screen of when electronic money is loaded to (put on) a wallet, an appointment screen of a medical institution, a progress screen of a medical treatment and various procedures at the medical institution (see FIG. 3 to FIG. 8), a confirmation screen of a prescription (see FIG. 6), a payment screen of when a payment of a medical fee is made, a confirmation screen of a medical statement and receipt, and the like.

More specifically, the display control unit 311 causes the operation/display unit 34 to display the progress information 232 of the user which information is acquired by the information acquisition unit 314. FIG. 3 to FIG. 8 are views illustrating examples of a progress information screen displayed on the operation/display unit 34 by the display control unit 311. As illustrated in the drawings, the progress information screen has a screen configuration in which a patient (user) name, a medical institution name, and a consultation appointment date and time at the medical institution are arranged on an upper side, and a current status is arranged on a lower side.

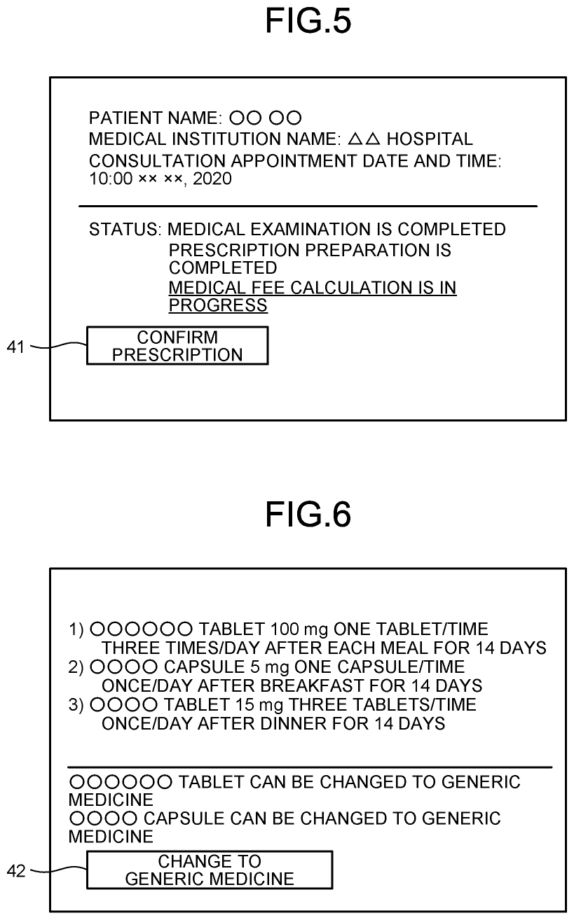

When the user is currently undergoing a medical examination, “status: under medical examination” is displayed, for example, as illustrated in FIG. 3.
Subsequently, when the medical examination of the user is completed, “status: medical examination is completed, and prescription preparation is in progress” is displayed, for example, as illustrated in FIG. 4. Subsequently, when the preparation of the prescription is completed, “status: medical examination is completed, prescription preparation is completed, and medical fee calculation is in progress” is displayed, for example, as illustrated in FIG. 5.

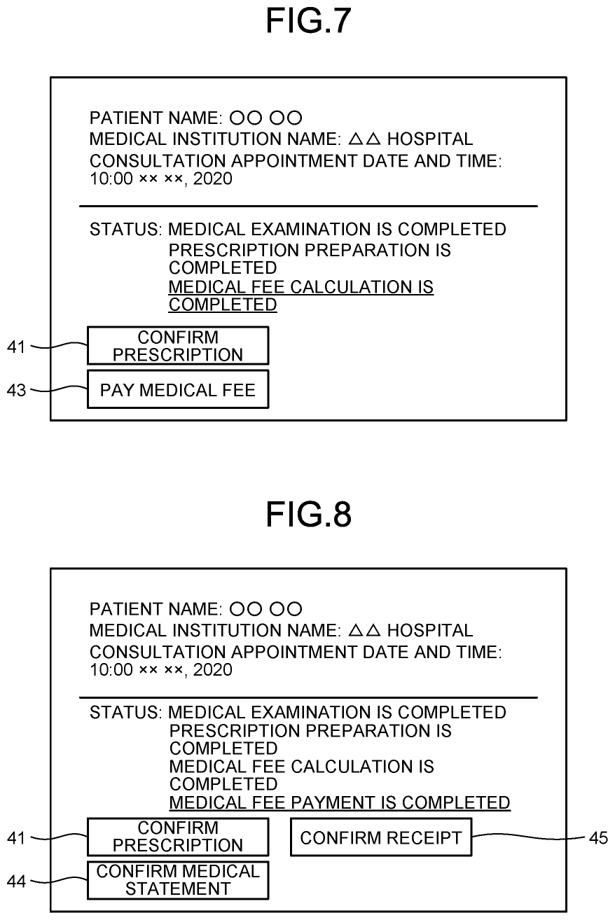

Subsequently, when the calculation of the medical fee is completed, “status: medical examination is completed, prescription preparation is completed, and medical fee calculation is completed” is displayed, for example, as illustrated in FIG. 7. Subsequently, when a payment of the medical fee is completed, “status: medical examination is completed, prescription preparation is completed, medical fee calculation is completed, and medical fee payment is completed” is displayed, for example, as illustrated in FIG. 8. In such a manner, new progress conditions are sequentially added to the progress information screen, and it is possible to see at a glance how far a medical treatment and various procedures of the user at the medical institution are progressed. Note that FIG. 4 to FIG. 8 are merely examples of a progress information screen, and a screen layout and order of adding progress conditions are not specifically limited.

Here, when causing the operation/display unit 34 to display the progress information 232 of the user, the display control unit 311 may display a button 41 to confirm a prescription, for example, as illustrated in FIG. 5. When the button 41 is pressed by the user, the display control unit 311 causes the operation/display unit 34 to display the prescription, for example, as illustrated in FIG. 6. As a result, since contents of the prescription may be quickly confirmed through the user terminal 3, convenience for the user is improved.

Moreover, when causing the operation/display unit 34 to display the progress information 232 of the user, the display control unit 311 may display a button (second button) 42 to change a prescription, for example, as illustrated in FIG. 6. Note that “changing contents of a prescription” specifically means changing a non-generic medicine included in the prescription to a generic medicine. When the button 42 is pressed by the user, the control unit 31 outputs a prescription change request for changing an already-prepared prescription to the medical institution server 2.

In response to this, the control unit 21 of the medical institution server 2 prepares new prescription data and performs an output thereof to the user terminal 3. Then, the display control unit 311 causes the operation/display unit 34 to display a new prescription. As a result, since it is not necessary to request a receptionist or a doctor at the medical institution to change a non-generic medicine included in the prescription to a generic medicine, convenience for the user is improved.

Note that, for example, as illustrated in FIG. 7, when a prescription is changed at a stage at which preparation of the prescription and calculation of a medical fee are completed, there is a case where the medical fee is changed from an initial amount. Thus, for example, when the button 42 is pressed by the user, the control unit 31 may output a recalculation request for recalculating the medical fee to the medical institution server 2 together with the prescription change request. In response to this, the control unit 21 of the medical institution server 2 prepares new medical fee data together with new prescription data and performs an output thereof to the user terminal 3. As a result, when medical fee is changed due to the change of the prescription, it is not necessary to give a request to the receptionist at the medical institution. Thus, the convenience for the user is improved.

Moreover, when causing the operation/display unit 34 to display the progress information 232 of the user, the display control unit 311 may display a button (first button) 43 for paying the medical fee as illustrated in FIG. 7. When the button 43 is pressed by the user, the payment processing unit 313 outputs, to the wallet server 1, a payment request for paying the medical fee with electronic money put on an electronic wallet, for example. Subsequently, the control unit 21 of the medical institution server 2 transfers the payment request acquired from the user terminal 3 to the wallet server 1.

In response to this, the control unit 11 of the wallet server 1 reduces a balance of the electronic money in the wallet of the user based on a payment amount included in the payment request. That is, the control unit 11 updates the wallet information 132 and the payment information 133 stored in the storage unit 13 according to a decreased amount of the balance of the electronic money in the wallet. Then, the control unit 11 outputs the updated wallet information 132 and payment information 133 to the user terminal 3. Furthermore, the control unit 11 outputs information indicating that the payment is completed (hereinafter, referred to as “payment completion information”) to the medical institution server 2. As a result, since it is possible to pay the medical fee on the spot without waiting for a long time at a reception desk of the medical institution, convenience for the user is improved.

Moreover, when causing the operation/display unit 34 to display the progress information 232 of the user, the display control unit 311 may display a button 44 for a confirmation of a medical statement and a button 45 for a confirmation of a receipt as illustrated in FIG. 8. When the buttons 44 and 45 are pressed by the user, the display control unit 311 causes the operation/display unit 34 to display the medical statement and the receipt.

The loading processing unit 312 loads electronic money to the wallet. The loading processing unit 312 outputs, to the wallet server 1, a loading request for causing a previously-set loading means to load electronic money to the wallet. Note that the “loading means” indicates a means to put electronic money on a wallet that is a virtual deposit/withdrawal account. Examples of the loading means include a bank account, a credit card, and the like.

The control unit 11 of the wallet server 1 that acquires the loading request from the loading processing unit 312 increases the balance of the electronic money in the wallet based on loaded amount information included in the loading request. That is, the control unit 11 updates the wallet information 132 stored in the storage unit 13 according to an increased amount of the balance of the electronic money in the wallet.

The payment processing unit 313 pays the medical fee to the medical institution. The payment processing unit 313 outputs, to the medical institution server 2, a payment request for making a payment by using, for example, the electronic money loaded on the wallet or other payment means (such as pre-registered bank account or credit card).

The control unit 21 of the medical institution server 2 that acquires a payment request from the user terminal 3 outputs the payment request including a payment amount (medical fee) to the wallet server 1 through the network NW. The control unit 11 of the wallet server 1 that acquires the payment request from the medical institution server 2 reduces the balance of the electronic money in the wallet of the user based on the payment amount included in the payment request, and outputs updated wallet information 132 and payment information 133 to the user terminal 3. Furthermore, the control unit 11 outputs payment completion information to the medical institution server 2.

The information acquisition unit 314 acquires the progress information 232 of a user from the medical institution server 2 when the user visits the medical institution. In response to this, the display control unit 311 causes the operation/display unit 34 to display the progress information 232 acquired by the information acquisition unit 314. Note that “when the user visits the medical institution” indicates a case where an arrival of the user at the medical institution is detected, for example, based on positional information of the user terminal 3 which information is detected by the positioning unit 35.

The information acquisition unit 314 may also acquire referral letter (medical information provision document) information of when a user is referred to another medical institution, for example. In this case, the information acquisition unit 314 acquires the referral letter information created based on a medical treatment history of the user at the medical institution from the medical institution server 2. In response to this, the control unit 31 outputs the referral letter information to a medical institution server 2 provided at a referral medical institution. As a result, since it is possible to acquire a referral letter without going directly to a medical institution, convenience for the user is improved.

Note that the control unit 31 may output appointment information for making an appointment at the referral medical institution together with the referral letter information to the medical institution server 2 provided at the referral medical institution. This appointment information is separately created by the control unit 31 on the basis, for example, of an operation on the user terminal 3 by the user.

Moreover, the information acquisition unit 314 may acquire the history information 231 of the user, for example. In this case, the information acquisition unit 314 acquires the history information 231 including a medical treatment history, medicine prescription history, and medical fee payment history of the user from the medical institution server 2. In response to this, the display control unit 311 causes the operation/display unit 34 to display the history information 231 acquired by the information acquisition unit 314. As a result, since it is possible to acquire a medical treatment history and the like of the user without directly going to a medical institution, convenience for the user is improved.

The storage unit 33 stores data of various screens displayed on the operation/display unit 34 by the display control unit 311. Moreover, in the storage unit 33, the user information 131, wallet information 132, payment information 133, positional information of the user terminal 3 which information is acquired by the positioning unit 35, history information 231 of the user which information is acquired from the medical institution server 2, progress information 232, referral letter information, and the like are stored when necessary.

The operation/display unit 34 includes, for example, a touch panel display, and has an input function of receiving an operation by a finger of a user, a pen, or the like, and a display function of displaying various kinds of information under the control by the display control unit 311.

The positioning unit 35 receives a radio wave from a global positioning system (GPS) satellite and detects positional information of the user terminal 3. Then, the control unit 31 stores, in the storage unit 33, the positional information of the user terminal 3 which information is detected by the positioning unit 35.

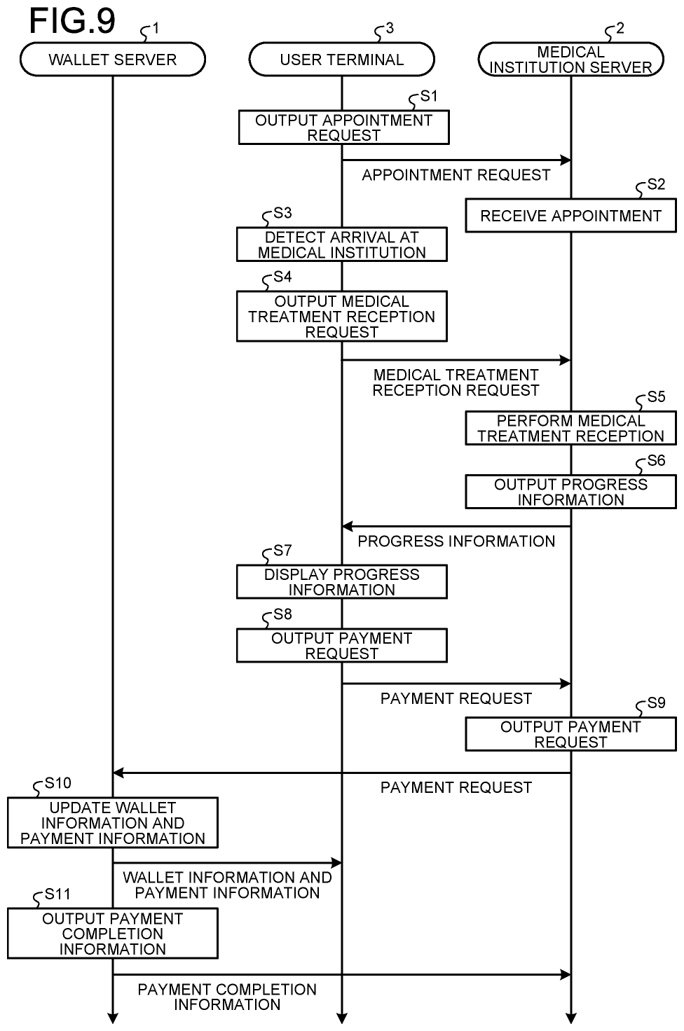

In the following, an example of a progress information displaying method of a medical institution which method is executed by the wallet system will be described with reference to FIG. 9. Note that in the following, the description will be made on the assumption of a situation in which a user makes an online appointment at a medical institution at home or the like and progress of a medical treatment and various procedures is displayed after an arrival at the medical institution.

First, a control unit 31 of a user terminal 3 outputs an appointment request for a medical institution to a medical institution server 2 (Step S1). Note that an appointment at a medical institution is made, for example, through a website or the like operated by the medical institution. Subsequently, a control unit 21 of the medical institution server 2 that acquires the appointment request receives the appointment for a user (Step S2).

Subsequently, the control unit 31 of the user terminal 3 detects that the user arrives at the medical institution (Step S3), and outputs a medical treatment reception request to the medical institution server 2 (Step S4). Note that in Step S3, it is detected that the user arrives at the medical institution based on positional information of the user terminal 3 which information is detected by a positioning unit 35.

Subsequently, the control unit 21 of the medical institution server 2 that acquires the medical treatment reception request performs medical treatment reception (Step S5). Subsequently, the control unit 21 of the medical institution server 2 outputs progress information 232 to the user terminal 3 every time a status is changed (Step S6).

Subsequently, a display control unit 311 of the user terminal 3 causes an operation/display unit 34 to display the acquired progress information 232 (Step S7). Subsequently, the display control unit 311 of the user terminal 3 outputs a payment request to the medical institution server 2 when a button 43 for paying a medical fee (see FIG. 7) is pressed by the user (Step S8). Subsequently, the control unit 21 of the medical institution server 2 outputs a payment request including a payment amount (medical fee) to a wallet server 1 (Step S9).

Subsequently, a control unit 11 of the wallet server 1 updates wallet information 132 and payment information 133 of the user based on the payment amount included in the payment request, and performs an output thereof to the user terminal 3 (Step S10). Subsequently, the control unit 11 outputs payment completion information to the medical institution server 2 (Step S11), and this flow is completed.

As described above, according to a mobile terminal, wallet program, and wallet system of the embodiment, an appointment of a medical treatment, payment of a medical fee, confirmation and change of a prescription, acquisition of a referral letter and medical treatment history, and the like may be performed online. Thus, it is possible to improve convenience for a user who uses a medical institution. Moreover, according to the mobile terminal, wallet program, and wallet system, it is possible to reduce anxiety of the user about waiting time at the medical institution by sequentially displaying progress conditions of a medical treatment and various procedures at the medical institution.

Further effects and modification examples may be easily derived by those skilled in the art. Accordingly, broader aspects of the present disclosure are not limited by the specific details and representative embodiments that are illustrated and described in the above manner. Thus, various modifications may be made without departing from the spirit or scope of a general concept of the disclosure defined by the accompanying claims and an equivalent thereof.

For example, in the mobile terminal, wallet program, and wallet system, when the button 42 for changing a prescription (see FIG. 6) is pressed by a user, a prescription change request is output to the medical institution server 2 and the prescription is changed on a side of the medical institution server 2. However, a prescription may be changed on a side of a user terminal 3. In this case, when a button 42 for changing a prescription is pressed by a user, a control unit 31 of a user terminal 3 rewrites a non-generic medicine included in the prescription to a previously-set generic medicine. Then, a display control unit 311 of the user terminal 3 causes an operation/display unit 34 to display the rewritten prescription. As a result, since the prescription may be changed on the side of the user terminal 3, convenience for the user is further improved.

According to the present disclosure, convenience for a user who uses a medical institution may be improved.

Although the disclosure has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A mobile terminal comprising:
a display; and
a processor comprising hardware, the processor being configured to
acquire progress information indicating progress of when a user visits a medical institution from a first server provided at the medical institution, and
control the display to display the progress information.

2. The mobile terminal according to claim 1, wherein the progress information includes information indicating whether the user is undergoing a medical examination, information indicating whether preparation of a prescription is completed, and information indicating whether a payment of a medical fee is completed.

3. The mobile terminal according to claim 1, wherein the processor is configured to
control the display to display a first button when controlling the display to display the progress information, and
output, when the first button is pressed by the user, a payment request for paying a medical fee with electronic money put on the electronic wallet to a second server configured to manage an electronic wallet.

4. The mobile terminal according to claim 1, wherein the processor is configured to
control the display to display a second button when causing the display to display the progress information, and
output a change request for changing an already-prepared prescription to the first server when the second button is pressed by the user.

5. The mobile terminal according to claim 4, wherein the processor is configured to output a recalculation request for recalculating a medical fee to the first server together with the change request of the prescription when the second button is pressed by the user.

6. The mobile terminal according to claim 1, wherein the processor is configured to
acquire referral letter information created based on a medical treatment history of the user at the medical institution from the first server, and
output the referral letter information to a first server provided at a referral medical institution.

7. The mobile terminal according to claim 1, wherein the processor is configured to
acquire history information including a medical treatment history, medicine prescription history, and medical fee payment history of the user at the medical institution from the first server, and
control the display to display the history information.

8. A non-transitory computer-readable recording medium on which an executable program is recorded, the program causing a processor of a computer to execute:
acquiring progress information indicating progress of when a user visits a medical institution from a first server provided at the medical institution; and
controlling a display included in a mobile terminal to display the progress information.

9. The non-transitory computer-readable recording medium according to claim 8, wherein the progress information includes information indicating whether the user is undergoing a medical examination, information indicating whether preparation of a prescription is completed, and information indicating whether a payment of a medical fee is completed.

10. The non-transitory computer-readable recording medium according to claim 8, wherein the program causes the processor to execute:
controlling the display to display a first button when controlling the display to display the progress information; and
outputting, when the first button is pressed by the user, a payment request for paying a medical fee with electronic money put on the electronic wallet to a second server configured to manage an electronic wallet.

11. The non-transitory computer-readable recording medium according to claim 8, wherein the program causes the processor to execute:
controlling the display to display a second button when controlling the display to display the progress information; and
outputting a change request for changing an already-prepared prescription to the first server when the second button is pressed by the user.

12. The non-transitory computer-readable recording medium according to claim 11, wherein the program causes the processor to execute outputting a recalculation request for recalculating a medical fee to the first server together with the change request of the prescription when the second button is pressed by the user.

13. The non-transitory computer-readable recording medium according to claim 8, wherein the program causes the processor to execute:
acquiring referral letter information created based on a medical treatment history of the user at the medical institution from the first server; and
outputting the referral letter information to a first server provided at a referral medical institution.

14. The non-transitory computer-readable recording medium according to claim 8, wherein the program causes the processor to execute:
acquiring history information including a medical treatment history, medicine prescription history, and medical fee payment history of the user at the medical institution from the first server; and
controlling the display to display the history information.

15. A wallet system comprising:
a first server provided at a medical institution and comprising a first processor comprising hardware;
a second server comprising a second processor comprising hardware and configured to manages an electronic wallet; and
a mobile terminal comprising
a display, and
a third processor comprising hardware, the third processor being configured to
acquire progress information indicating progress of when a user visits the medical institution from the first server, and
control the display to display the progress information.

16. The wallet system according to claim 15, wherein the progress information includes information indicating whether the user is undergoing a medical examination, information indicating whether preparation of a prescription is completed, and information indicating whether a payment of a medical fee is completed.

17. The wallet system according to claim 15, wherein the third processor is configured to:
control the display to display a first button when controlling the display to display the progress information; and
output, when the first button is pressed by the user, a payment request for paying a medical fee with electronic money put on the electronic wallet to the second server.

18. The wallet system according to claim 15, wherein the third processor is configured to:
control the display to display a second button when controlling the display to display the progress information, and
output a change request for changing an already-prepared prescription to the first server when the second button is pressed by the user.

19. The wallet system according to claim 18, wherein the third processor is configured to output a recalculation request for recalculating a medical fee to the first server together with the change request of the prescription when the second button is pressed by the user.

20. The wallet system according to claim 15, wherein the third processor is configured to
acquire referral letter information created based on a medical treatment history of the user at the medical institution from the first server, and
output the referral letter information to a first server provided at a referral medical institution.
